(19) United States
(12) Patent Application Publication
(10) Pub. No.: US 2022/0215477 A1
(43) Pub. Date: Jul. 7, 2022

(54) PROCESSING WORK BENCH

(71) Applicant: Cigna Intellectual Property, Inc., Wilmington, DE (US)

(72) Inventors: Jeff L. White, Pensacola, FL (US); Ann Cooper, Fuquay Varina, NC (US); Danielle M. Palumbo, South Abington Township, PA (US); Michael Sterni, Totowa, NJ (US); Krishna K. Ponugoti, Windsor Locks, CT (US)

(21) Appl. No.: 17/141,496

(22) Filed: Jan. 5, 2021

(51) Int. Cl. G06Q 40/08; G06Q 10/10; G16H 40/00

(52) U.S. Cl. CPC G06Q 40/08; G16H 40/00; G06Q 10/10

(57) ABSTRACT

A processing system includes: a type module configured to receive a claim from a claim engine and to determine a type of the claim based on the claim engine; an inclusions and exclusions module configured to, based on the type of the claim, select inclusions and exclusions for the claim; a transmitting module configured to transmit, to a claim processing module, the claim, the selected inclusions and exclusions, and the type of the claim; and a responding module configured to: receive, from the claim processing module, a response including an indicator of whether to approve or deny the claim, the response being determined by the claim processing module based on the claim, the selected inclusions and exclusions, the type of the claim, and rules regarding approving and denying claims; and to respond to the claim engine with the indicator of whether to approve or deny the claim.

### FIELD

The present disclosure relates to processing systems and methods and more particularly to systems and methods for processing claims.

### BACKGROUND

People visit health care providers for various reasons, such as when they are sick, for regular checkups, etc. Health care providers submit claims for their interactions with individuals. Each claim is for medical services provided to an individual. Each claim may include one or more procedure codes and other information regarding treatment provided to an individual.

Claims may be sent to a claim processor. The claim processor may check claims for completeness and accuracy and determine whether the services provided are covered under a benefit plan of an individual. The claim processor may initiate payment (e.g., by an insurance provider) for services that are covered under the individual's benefit plan.

The claim processor may also verify other information regarding a claim, such as a co-pay of an individual, any deductible of the individual, maximum out of pocket of an individual, etc. Once a claim is processed, an individual may be sent an explanation of benefits (EOB). The EOB may include details of the services received and indicate whether services perform are or are not covered under the individual's benefit plan.

The background description provided here is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### SUMMARY

In a feature, a processing system includes: a type module configured to receive a claim from a claim engine and to determine a type of the claim based on the claim engine from which the claim was received; an inclusions and exclusions module configured to, based on the type of the claim, select inclusions and exclusions for the claim; a transmitting module configured to transmit, to a claim processing module, (a) the claim, (b) the selected inclusions and exclusions, and (c) the type of the claim; and a responding module configured to: receive, from the claim processing module, a response including an indicator of whether to (a) approve the claim or (b) deny the claim, the response being determined by the claim processing module based on the claim, the selected inclusions and exclusions, the type of the claim, and rules regarding approving and denying claims; and to respond to the claim engine with the indicator of whether to (a) approve the claim or (b) deny the claim.

In further features, the type is selected from a group consisting of: governmental and non-governmental.

In further features, the type module is configured to: set the type to governmental when the claim engine is included in a first predetermined set of claim engines; and set the type to non-governmental when the claim engine is included in a second predetermined set of claim engines, where none of the claim engines in the second predetermined set of claim engines is included in the first predetermined set of claim engines.

In further features, the inclusions and exclusions module is configured to: select first inclusions and first exclusions when the type of the claim is governmental; and select second inclusions and second exclusions when the type of the claim is non-governmental.

In further features: at least one of the first inclusions is not included in the second inclusions; and at least one of the second inclusions is not included in the first inclusions.

In further features: at least one of the first exclusions is not included in the second exclusions; and at least one of the second exclusions is not included in the first exclusions.

In further features, the transmitting module is configured to transmit (a) the claim, (b) the selected inclusions and exclusions, and (c) the type of the claim to the claim processing module via a network.

In further features, the type module is configured to receive the claim from the claim engine via a network.

In further features, a storage module is configured to store the claim in memory, to store the type of the claim in the memory in association with the claim, and to store, in association with the claim, the indicator of whether to (a) approve the claim or (b) deny the claim.

In further features, the claim includes a claim code for a healthcare procedure performed by a health care provider.

In further features, the claim processing module includes the ClaimsXten™ claim processing module.

In further features, the selected inclusions include rules for denying claims and the selected exclusions include rules for approving claims.

In a feature, a processing method includes: receiving a claim from a claim engine; determining a type of the claim based on the claim engine from which the claim was received; based on the type of the claim, selecting inclusions and exclusions for the claim; transmitting, to a claim processing module, (a) the claim, (b) the selected inclusions and exclusions, and (c) the type of the claim; receiving, from the claim processing module, a response including an indicator of whether to (a) approve the claim or (b) deny the claim, the response being determined by the claim processing module based on the claim, the selected inclusions and exclusions, the type of the claim, and rules regarding approving and denying claims; and responding to the claim engine with the indicator of whether to (a) approve the claim or (b) deny the claim.

In further features, the type is selected from a group consisting of: governmental and non-governmental.

In further features, the processing method further includes: setting the type to governmental when the claim engine is included in a first predetermined set of claim engines; and setting the type to non-governmental when the claim engine is included in a second predetermined set of claim engines, where none of the claim engines in the second predetermined set of claim engines is included in the first predetermined set of claim engines.

In further features, the selecting inclusions and exclusions includes: selecting first inclusions and first exclusions when the type of the claim is governmental; and selecting second inclusions and second exclusions when the type of the claim is non-governmental.

In further features: at least one of the first inclusions is not included in the second inclusions; and at least one of the second inclusions is not included in the first inclusions.

In further features: at least one of the first exclusions is not included in the second exclusions; and at least one of the second exclusions is not included in the first exclusions.

In further features, the processing method further includes: storing the claim in memory; storing the type of the claim in the memory in association with the claim; and storing, in association with the claim, the indicator of whether to (a) approve the claim or (b) deny the claim.

In further features, the selected inclusions include rules for denying claims and the selected exclusions include rules for approving claims.

Further areas of applicability of the present disclosure will become apparent from the detailed description, the claims, and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings.

FIG. 1 is a functional block diagram of an example system including a high-volume pharmacy.

FIG. 2 is a functional block diagram of an example pharmacy fulfillment device, which may be deployed within the system of FIG. 1.

FIG. 3 is a functional block diagram of an example order processing device, which may be deployed within the system of FIG. 1.

FIG. 4 is a functional block diagram of an example implementation of a claim processing system.

FIG. 5 is a functional block diagram of an example implementation of the benefit manager device.

FIG. 6 is a flowchart depicting an example method of processing a claim.

FIG. 7 is an example table including claim identifiers, action dates, action types, adjustment codes, references, and explanations for inclusions.

FIG. 8 is an example table including claim identifiers, action dates, action types, adjustment codes, references, and explanations for inclusions.

In the drawings, reference numbers may be reused to identify similar and/or identical elements.

### DETAILED DESCRIPTION

Claims (e.g., medical, pharmacy, etc.) may be received from multiple different claim engines. The claims may be logged before being forwarded to a claim processor, which determines whether to approve or deny claims. The decisions regarding whether to approve or deny claims are logged with the claims and transmitted to the claim engines from which the claims, respectively, were received.

The present application involves determining a type of a claim received from a claim engine. For example, a claim may be determined to be a governmental claim if the claim is received from one of a set of governmental claim engines and determined to be a non-governmental claim otherwise.

Different inclusions and exclusions may be used by the claim processor depending on the type of the claim. The present application involves transmitting the inclusions and exclusions specific to the claim type to the claim processor for use in determining whether to approve or deny the claim. This may make approval/denial decisions by the claim processor more consistent and decrease the period for the claim processor to make a decision regarding whether to approve or deny a claim. Throughput and response time of the claim processor may therefore be increased. The inclusions and exclusions can also be adjusted, such as by a benefit plan provider.

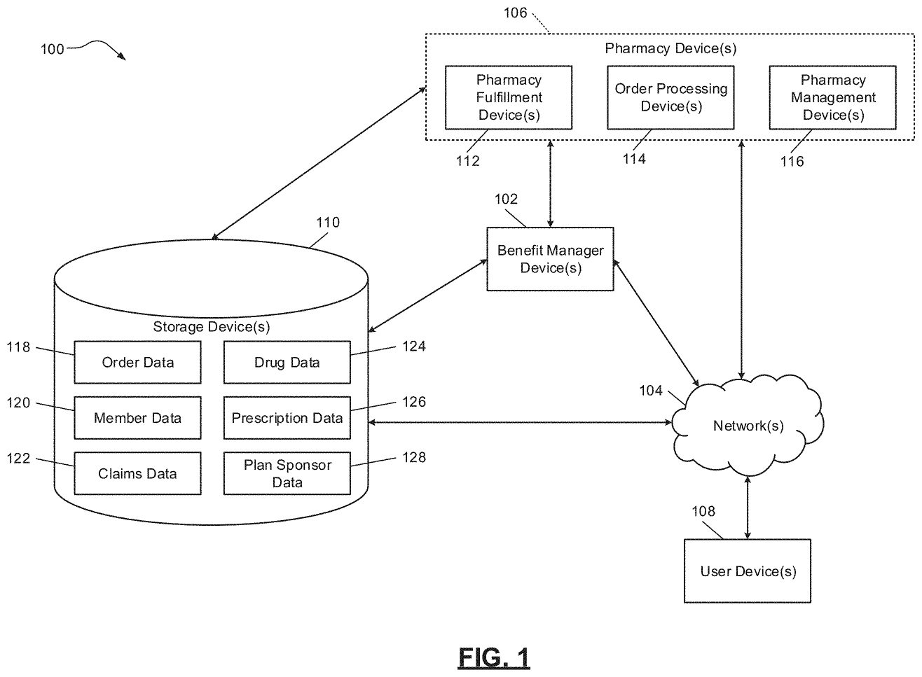

FIG. 1 is a block diagram of an example implementation of a system 100 for a high-volume pharmacy. While the example of a pharmacy and pharmacy related claims is provided, the present application is also applicable to non-pharmacy claims. While the system 100 is generally described as being deployed in a high-volume pharmacy or a fulfillment center (for example, a mail order pharmacy, a direct delivery pharmacy, etc.), the system 100 and/or components of the system 100 may otherwise be deployed (for example, in a lower-volume pharmacy, etc.). A high-volume pharmacy may be a pharmacy that is capable of filling at least some prescriptions mechanically. The system 100 may include a reimbursement manager device 406 and a pharmacy device 106 in communication with each other directly and/or over a network 104.

The system 100 may also include one or more user device(s) 108. A user, such as a pharmacist, patient, data analyst, health plan administrator, etc., may access the benefit manager device 102 or the pharmacy device 106 using the user device 108. The user device 108 may be a desktop computer, a laptop computer, a tablet, a smartphone, etc.

The benefit manager device 102 is a device operated by an entity that is at least partially responsible for creation and/or management of the pharmacy or drug benefit and/or health benefits, etc. While the entity operating the benefit manager device 102 may be a pharmacy benefit manager (PBM), other entities may operate the benefit manager device 102 on behalf of themselves or other entities. For example, the benefit manager device 102 may be operated by a health plan, a retail pharmacy chain, a drug wholesaler, a data analytics or other type of software-related company, etc. In some implementations, a PBM that provides the pharmacy benefit may provide one or more additional benefits including a medical or health benefit, a dental benefit, a vision benefit, a wellness benefit, a radiology benefit, a pet care benefit, an insurance benefit, a long term care benefit, a nursing home benefit, etc. The PBM may, in addition to its PBM operations, operate one or more pharmacies. The pharmacies may be retail pharmacies, mail order pharmacies, etc.

Some of the operations of a PBM that operates the benefit manager device 102 may include the following activities and processes. A member (or a person on behalf of the member) of a pharmacy benefit plan may obtain a prescription drug at a retail pharmacy location (e.g., a location of a physical store) from a pharmacist or a pharmacist technician. The member may also obtain the prescription drug through mail order drug delivery from a mail order pharmacy location, such as the system 100. In some implementations, the member may obtain the prescription drug directly or indirectly through the use of a machine, such as a kiosk, a vending unit, a mobile electronic device, or a different type of mechanical device, electrical device, electronic communication device, and/or computing device. Such a machine may be filled with the prescription drug in prescription packaging, which may include multiple prescription components, by the system 100. The pharmacy benefit plan and/or other types of benefit plans may be administered by or through the benefit manager device 102.

The member may have a copayment for the prescription drug that reflects an amount of money that the member is responsible to pay the pharmacy for the prescription drug. The money paid by the member to the pharmacy may come from, as examples, personal funds of the member, a health savings account (HSA) of the member or the member's family, a health reimbursement arrangement (HRA) of the member or the member's family, or a flexible spending account (FSA) of the member or the member's family. In some instances, an employer of the member may directly or indirectly fund or reimburse the member for the copayments.

The amount of the copayment required by the member may vary across different pharmacy benefit plans having different plan sponsors or clients and/or for different prescription drugs. The member's copayment may be a flat copayment (in one example, $10), coinsurance (in one example, 10%), and/or a deductible (for example, responsibility for the first $500 of annual prescription drug expense, etc.) for certain prescription drugs, certain types and/or classes of prescription drugs, and/or all prescription drugs. The copayment may be stored in a storage device 110 or determined by the benefit manager device 102.

In some instances, the member may not pay the copayment or may only pay a portion of the copayment for the prescription drug. For example, if an usual and customary cost for a generic version of a prescription drug is $4, and the member's flat copayment is $20 for the prescription drug, the member may only need to pay $4 to receive the prescription drug. In another example involving a worker's compensation claim, no copayment may be due by the member for the prescription drug.

In addition, copayments may also vary based on different delivery channels for the prescription drug. For example, the copayment for receiving the prescription drug from a mail order pharmacy location may be less than the copayment for receiving the prescription drug from a retail pharmacy location.

In conjunction with receiving a copayment (if any) from the member and dispensing the prescription drug to the member, the pharmacy submits a claim to the PBM for the prescription drug. After receiving the claim, the PBM (such as by using the benefit manager device 102) may perform certain adjudication operations including verifying eligibility for the member, identifying/reviewing an applicable formulary for the member to determine any appropriate copayment, coinsurance, and deductible for the prescription drug, and performing a drug utilization review (DUR) for the member. Further, the PBM may provide a response to the pharmacy (for example, the system 100) following performance of at least some of the aforementioned operations.

As part of the adjudication, a plan sponsor (or the benefit manager on behalf of the plan sponsor) ultimately reimburses the pharmacy for filling the prescription drug when the prescription drug was successfully adjudicated. The aforementioned adjudication operations generally occur before the copayment is received and the prescription drug is dispensed. However in some instances, these operations may occur simultaneously, substantially simultaneously, or in a different order. In addition, more or fewer adjudication operations may be performed as at least part of the adjudication process.

The amount of reimbursement paid by a plan sponsor and/or money paid by the member may be determined at least partially based on types of networks in which the benefit manager is included. In some implementations, the amount may also be determined based on other factors. For example, if the member pays for the prescription drug without using the prescription or drug benefit provided by the PBM, the amount of money paid by the member may be higher than when the member uses the prescription or drug benefit. In some implementations, the amount of money received by the pharmacy for dispensing the prescription drug and for the prescription drug itself may be higher than when the member uses the prescription or drug benefit. Some or all of the foregoing operations may be performed by executing instructions stored in the benefit manager device 102 and/or an additional device. The benefit manager device 102 is discussed further below.

Examples of the network 104 include a Global System for Mobile Communications (GSM) network, a code division multiple access (CDMA) network, 3rd Generation Partnership Project (3GPP), an Internet Protocol (IP) network, a Wireless Application Protocol (WAP) network, or an IEEE 802.11 standards network, as well as various combinations of the above networks. The network 104 may include an optical network. The network 104 may be a local area network or a global communication network, such as the Internet. In some implementations, the network 104 may include a network dedicated to prescription orders: a prescribing network such as the electronic prescribing network operated by Surescripts of Arlington, Va.

Moreover, although the system shows a single network 104, multiple networks can be used. The multiple networks may communicate in series and/or parallel with each other to link the devices 102-110.

The pharmacy device 106 may be a device associated with a retail pharmacy location (e.g., an exclusive pharmacy location, a grocery store with a retail pharmacy, or a general sales store with a retail pharmacy) or other type of pharmacy location at which a member attempts to obtain a prescription. The pharmacy may use the pharmacy device 106 to submit the claim to the PBM for adjudication.

Additionally, in some implementations, the pharmacy device 106 may enable information exchange between the pharmacy and the PBM. For example, this may allow the sharing of member information such as drug history that may allow the pharmacy to better service a member (for example, by providing more informed therapy consultation and drug interaction information). In some implementations, the benefit manager device 102 may track prescription drug fulfillment and/or other information for users that are not members, or have not identified themselves as members, at the time (or in conjunction with the time) in which they seek to have a prescription filled at a pharmacy.

The pharmacy device 106 may include a pharmacy fulfillment device 112, an order processing device 114, and a pharmacy management device 116 in communication with each other directly and/or over the network 104. The order processing device 114 may receive information regarding filling prescriptions and may direct an order component to one or more devices of the pharmacy fulfillment device 112 at a pharmacy. The pharmacy fulfillment device 112 may fulfill, dispense, aggregate, and/or pack the order components of the prescription drugs in accordance with one or more prescription orders directed by the order processing device 114.

In general, the order processing device 114 is a device located within or otherwise associated with the pharmacy to enable the pharmacy fulfilment device 112 to fulfill a prescription and dispense prescription drugs. In some implementations, the order processing device 114 may be an external order processing device separate from the pharmacy and in communication with other devices located within the pharmacy.

For example, the external order processing device may communicate with an internal pharmacy order processing device and/or other devices located within the system 100. In some implementations, the external order processing device may have limited functionality (e.g., as operated by a user requesting fulfillment of a prescription drug), while the internal pharmacy order processing device may have greater functionality (e.g., as operated by a pharmacist).

The order processing device 114 may track the prescription order as it is fulfilled by the pharmacy fulfillment device 112. The prescription order may include one or more prescription drugs to be filled by the pharmacy. The order processing device 114 may make pharmacy routing decisions and/or order consolidation decisions for the particular prescription order. The pharmacy routing decisions include what device(s) in the pharmacy are responsible for filling or otherwise handling certain portions of the prescription order. The order consolidation decisions include whether portions of one prescription order or multiple prescription orders should be shipped together for a user or a user family. The order processing device 114 may also track and/or schedule literature or paperwork associated with each prescription order or multiple prescription orders that are being shipped together. In some implementations, the order processing device 114 may operate in combination with the pharmacy management device 116.

The order processing device 114 may include circuitry, a processor, a memory to store data and instructions, and communication functionality. The order processing device 114 is dedicated to performing processes, methods, and/or instructions described in this application. Other types of electronic devices may also be used that are specifically configured to implement the processes, methods, and/or instructions described in further detail below.

In some implementations, at least some functionality of the order processing device 114 may be included in the pharmacy management device 116. The order processing device 114 may be in a client-server relationship with the pharmacy management device 116, in a peer-to-peer relationship with the pharmacy management device 116, or in a different type of relationship with the pharmacy management device 116. The order processing device 114 and/or the pharmacy management device 116 may communicate directly (for example, such as by using a local storage) and/or through the network 104 (such as by using a cloud storage configuration, software as a service, etc.) with the storage device 110.

The storage device 110 may include: non-transitory storage (for example, memory, hard disk, CD-ROM, etc.) in communication with the benefit manager device 102 and/or the pharmacy device 106 directly and/or over the network 104. The non-transitory storage may store order data 118, member data 120, claims data 122, drug data 124, prescription data 126, and/or plan sponsor data 128. Further, the system 100 may include additional devices, which may communicate with each other directly or over the network 104.

The order data 118 may be related to a prescription order. The order data may include type of the prescription drug (for example, drug name and strength) and quantity of the prescription drug. The order data 118 may also include data used for completion of the prescription, such as prescription materials. In general, prescription materials include an electronic copy of information regarding the prescription drug for inclusion with or otherwise in conjunction with the fulfilled prescription. The prescription materials may include electronic information regarding drug interaction warnings, recommended usage, possible side effects, expiration date, date of prescribing, etc. The order data 118 may be used by a high-volume fulfillment center to fulfill a pharmacy order.

In some implementations, the order data 118 includes verification information associated with fulfillment of the prescription in the pharmacy. For example, the order data 118 may include videos and/or images taken of (i) the prescription drug prior to dispensing, during dispensing, and/or after dispensing, (ii) the prescription container (for example, a prescription container and sealing lid, prescription packaging, etc.) used to contain the prescription drug prior to dispensing, during dispensing, and/or after dispensing, (iii) the packaging and/or packaging materials used to ship or otherwise deliver the prescription drug prior to dispensing, during dispensing, and/or after dispensing, and/or (iv) the fulfillment process within the pharmacy. Other types of verification information such as barcode data read from pallets, bins, trays, or carts used to transport prescriptions within the pharmacy may also be stored as order data 118.

The member data 120 includes information regarding the members. The information stored as member data 120 may include personal information, personal health information, protected health information, etc. Examples of the member data 120 include name, address, telephone number, e-mail address, prescription drug history, etc. The member data 120 may include a plan sponsor identifier that identifies the plan sponsor associated with the member and/or a member identifier that identifies the member to the plan sponsor. The member data 120 may include a member identifier that identifies the plan sponsor associated with the user and/or a user identifier that identifies the user to the plan sponsor. The member data 120 may also include dispensation preferences such as type of label, type of cap, message preferences, language preferences, etc.

The member data 120 may be accessed by various devices in the pharmacy (for example, the high-volume fulfillment center, etc.) to obtain information used for fulfillment and shipping of prescription orders. In some implementations, an external order processing device operated by or on behalf of a member may have access to at least a portion of the member data 120 for review, verification, or other purposes.

In some implementations, the member data 120 may include information for persons who are users of the pharmacy but are not members of a benefit plan provided by a benefit provider. For example, these users may obtain drugs directly from the pharmacy, through a private label service offered by the pharmacy, the high-volume fulfillment center, or otherwise. In general, the terms “member” and “user” may be used interchangeably. For a prescription drug order, the member data 120 and the order data 118 may be associated in memory for the order.

The claims data 122 includes information regarding pharmacy claims adjudicated by the PBM under a drug benefit program provided by the PBM for one or more plan sponsors. In general, the claims data 122 includes an identification of the client that sponsors the drug benefit program under which the claim is made, and/or the member that purchased the prescription drug giving rise to the claim, the prescription drug that was filled by the pharmacy (e.g., the national drug code number, etc.), the dispensing date, generic indicator, generic product identifier (GPI) number, medication class, the cost of the prescription drug provided under the drug benefit program, the copayment/coinsurance amount, rebate information, and/or member eligibility, etc. Additional information may be included.

In some implementations, other types of claims beyond prescription drug claims may be stored in the claims data 122. For example, medical claims, dental claims, wellness claims, or other types of health-care-related claims for members may be stored as a portion of the claims data 122.

In some implementations, the claims data 122 includes claims that identify the members with whom the claims are associated. Additionally or alternatively, the claims data 122 may include claims that have been de-identified (that is, associated with a unique identifier but not with a particular, identifiable member).

The drug data 124 may include drug name (e.g., technical name and/or common name), other names by which the drug is known, active ingredients, an image of the drug (such as in pill form), etc. The drug data 124 may include information associated with a single medication or multiple medications.

The prescription data 126 may include information regarding prescriptions that may be issued by prescribers on behalf of users, who may be members of the pharmacy benefit plan—for example, to be filled by a pharmacy. Examples of the prescription data 126 include user names, medication or treatment (such as lab tests), dosing information, etc. The prescriptions may include electronic prescriptions or paper prescriptions that have been scanned. In some implementations, the dosing information reflects a frequency of use (e.g., once a day, twice a day, before each meal, etc.) and a duration of use (e.g., a few days, a week, a few weeks, a month, etc.).

In some implementations, the order data 118 may be linked to associated member data 120, claims data 122, drug data 124, and/or prescription data 126.

The plan sponsor data 128 includes information regarding the plan sponsors of the PBM. Examples of the plan sponsor data 128 include company name, company address, contact name, contact telephone number, contact e-mail address, etc.

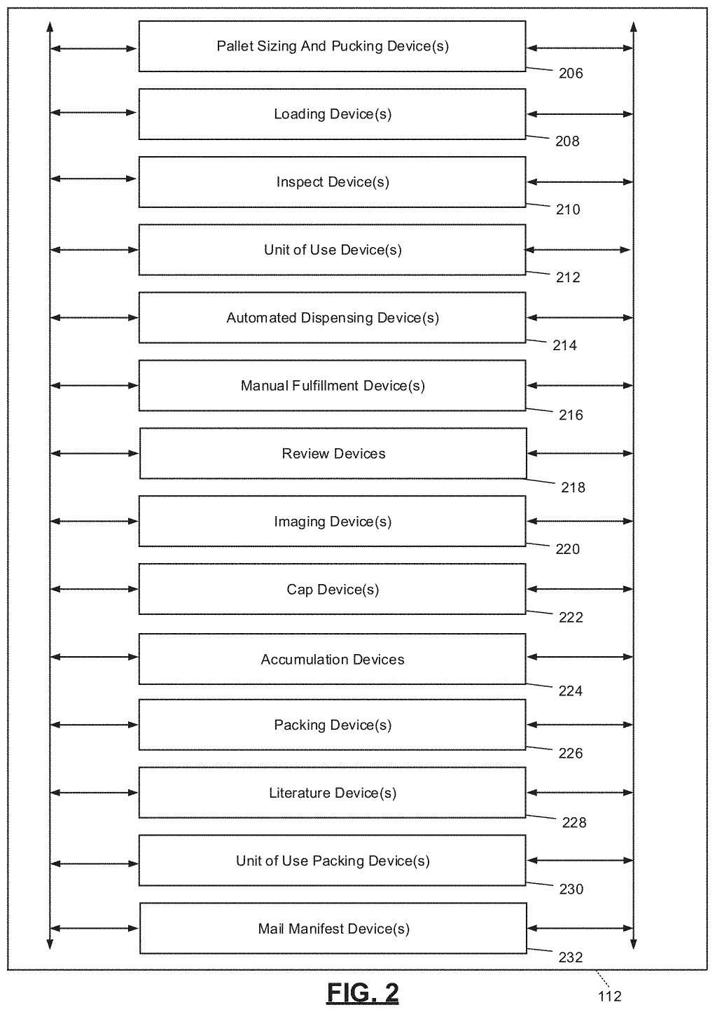

FIG. 2 illustrates the pharmacy fulfillment device 112 according to an example implementation. The pharmacy fulfillment device 112 may be used to process and fulfill prescriptions and prescription orders. After fulfillment, the fulfilled prescriptions are packed for shipping.

The pharmacy fulfillment device 112 may include devices in communication with the benefit manager device 102, the order processing device 114, and/or the storage device 110, directly or over the network 104. Specifically, the pharmacy fulfillment device 112 may include pallet sizing and pucking device(s) 206, loading device(s) 208, inspect device(s) 210, unit of use device(s) 212, automated dispensing device(s) 214, manual fulfillment device(s) 216, review devices 218, imaging device(s) 220, cap device(s) 222, accumulation devices 224, packing device(s) 226, literature device(s) 228, unit of use packing device(s) 230, and mail manifest device(s) 232. Further, the pharmacy fulfillment device 112 may include additional devices, which may communicate with each other directly or over the network 104.

In some implementations, operations performed by one of these devices 206-232 may be performed sequentially, or in parallel with the operations of another device as may be coordinated by the order processing device 114. In some implementations, the order processing device 114 tracks a prescription with the pharmacy based on operations performed by one or more of the devices 206-232.

In some implementations, the pharmacy fulfillment device 112 may transport prescription drug containers, for example, among the devices 206-232 in the high-volume fulfillment center, by use of pallets. The pallet sizing and pucking device 206 may configure pucks in a pallet. A pallet may be a transport structure for a number of prescription containers, and may include a number of cavities. A puck may be placed in one or more than one of the cavities in a pallet by the pallet sizing and pucking device 206. The puck may include a receptacle sized and shaped to receive a prescription container. Such containers may be supported by the pucks during carriage in the pallet. Different pucks may have differently sized and shaped receptacles to accommodate containers of differing sizes, as may be appropriate for different prescriptions.

The arrangement of pucks in a pallet may be determined by the order processing device 114 based on prescriptions that the order processing device 114 decides to launch. The arrangement logic may be implemented directly in the pallet sizing and pucking device 206. Once a prescription is set to be launched, a puck suitable for the appropriate size of container for that prescription may be positioned in a pallet by a robotic arm or pickers. The pallet sizing and pucking device 206 may launch a pallet once pucks have been configured in the pallet.

The loading device 208 may load prescription containers into the pucks on a pallet by a robotic arm, a pick and place mechanism (also referred to as pickers), etc. In various implementations, the loading device 208 has robotic arms or pickers to grasp a prescription container and move it to and from a pallet or a puck. The loading device 208 may also print a label that is appropriate for a container that is to be loaded onto the pallet, and apply the label to the container. The pallet may be located on a conveyor assembly during these operations (e.g., at the high-volume fulfillment center, etc.).

The inspect device 210 may verify that containers in a pallet are correctly labeled and in the correct spot on the pallet. The inspect device 210 may scan the label on one or more containers on the pallet. Labels of containers may be scanned or imaged in full or in part by the inspect device 210. Such imaging may occur after the container has been lifted out of its puck by a robotic arm, picker, etc., or may be otherwise scanned or imaged while retained in the puck. In some implementations, images and/or video captured by the inspect device 210 may be stored in the storage device 110 as order data 118.

The unit of use device 212 may temporarily store, monitor, label, and/or dispense unit of use products. In general, unit of use products are prescription drug products that may be delivered to a user or member without being repackaged at the pharmacy. These products may include pills in a container, pills in a blister pack, inhalers, etc. Prescription drug products dispensed by the unit of use device 212 may be packaged individually or collectively for shipping, or may be shipped in combination with other prescription drugs dispensed by other devices in the high-volume fulfillment center.

At least some of the operations of the devices 206-232 may be directed by the order processing device 114. For example, the manual fulfillment device 216, the review device 218, the automated dispensing device 214, and/or the packing device 226, etc. may receive instructions provided by the order processing device 114.

The automated dispensing device 214 may include one or more devices that dispense prescription drugs or pharmaceuticals into prescription containers in accordance with one or multiple prescription orders. In general, the automated dispensing device 214 may include mechanical and electronic components with, in some implementations, software and/or logic to facilitate pharmaceutical dispensing that would otherwise be performed in a manual fashion by a pharmacist and/or pharmacist technician. For example, the automated dispensing device 214 may include high-volume fillers that fill a number of prescription drug types at a rapid rate and blister pack machines that dispense and pack drugs into a blister pack. Prescription drugs dispensed by the automated dispensing devices 214 may be packaged individually or collectively for shipping, or may be shipped in combination with other prescription drugs dispensed by other devices in the high-volume fulfillment center.

The manual fulfillment device 216 controls how prescriptions are manually fulfilled. For example, the manual fulfillment device 216 may receive or obtain a container and enable fulfillment of the container by a pharmacist or pharmacy technician. In some implementations, the manual fulfillment device 216 provides the filled container to another device in the pharmacy fulfillment devices 112 to be joined with other containers in a prescription order for a user or member.

In general, manual fulfillment may include operations at least partially performed by a pharmacist or a pharmacy technician. For example, a person may retrieve a supply of the prescribed drug, may make an observation, may count out a prescribed quantity of drugs and place them into a prescription container, etc. Some portions of the manual fulfillment process may be automated by use of a machine. For example, counting of capsules, tablets, or pills may be at least partially automated (such as through use of a pill counter). Prescription drugs dispensed by the manual fulfillment device 216 may be packaged individually or collectively for shipping, or may be shipped in combination with other prescription drugs dispensed by other devices in the high-volume fulfillment center.

The review device 218 may process prescription containers to be reviewed by a pharmacist for proper pill count, exception handling, prescription verification, etc. Fulfilled prescriptions may be manually reviewed and/or verified by a pharmacist, as may be required by state or local law. A pharmacist or other licensed pharmacy person who may dispense certain drugs in compliance with local and/or other laws may operate the review device 218 and visually inspect a prescription container that has been filled with a prescription drug. The pharmacist may review, verify, and/or evaluate drug quantity, drug strength, and/or drug interaction concerns, or otherwise perform pharmacist services. The pharmacist may also handle containers which have been flagged as an exception, such as containers with unreadable labels, containers for which the associated prescription order has been canceled, containers with defects, etc. In an example, the manual review can be performed at a manual review station.

The imaging device 220 may image containers once they have been filled with pharmaceuticals. The imaging device 220 may measure a fill height of the pharmaceuticals in the container based on the obtained image to determine if the container is filled to the correct height given the type of pharmaceutical and the number of pills in the prescription. Images of the pills in the container may also be obtained to detect the size of the pills themselves and markings thereon. The images may be transmitted to the order processing device 114 and/or stored in the storage device 110 as part of the order data 118.

The cap device 222 may be used to cap or otherwise seal a prescription container. In some implementations, the cap device 222 may secure a prescription container with a type of cap in accordance with a user preference (e.g., a preference regarding child resistance, etc.), a plan sponsor preference, a prescriber preference, etc. The cap device 222 may also etch a message into the cap, although this process may be performed by a subsequent device in the high-volume fulfillment center.

The accumulation device 224 accumulates various containers of prescription drugs in a prescription order. The accumulation device 224 may accumulate prescription containers from various devices or areas of the pharmacy. For example, the accumulation device 224 may accumulate prescription containers from the unit of use device 212, the automated dispensing device 214, the manual fulfillment device 216, and the review device 218. The accumulation device 224 may be used to group the prescription containers prior to shipment to the member.

The literature device 228 prints, or otherwise generates, literature to include with each prescription drug order. The literature may be printed on multiple sheets of substrates, such as paper, coated paper, printable polymers, or combinations of the above substrates. The literature printed by the literature device 228 may include information required to accompany the prescription drugs included in a prescription order, other information related to prescription drugs in the order, financial information associated with the order (for example, an invoice or an account statement), etc.

In some implementations, the literature device 228 folds or otherwise prepares the literature for inclusion with a prescription drug order (e.g., in a shipping container). In other implementations, the literature device 228 prints the literature and is separate from another device that prepares the printed literature for inclusion with a prescription order.

The packing device 226 packages the prescription order in preparation for shipping the order. The packing device 226 may box, bag, or otherwise package the fulfilled prescription order for delivery. The packing device 226 may further place inserts (e.g., literature or other papers, etc.) into the packaging received from the literature device 228. For example, bulk prescription orders may be shipped in a box, while other prescription orders may be shipped in a bag, which may be a wrap seal bag.

The packing device 226 may label the box or bag with an address and a recipient's name. The label may be printed and affixed to the bag or box, be printed directly onto the bag or box, or otherwise associated with the bag or box. The packing device 226 may sort the box or bag for mailing in an efficient manner (e.g., sort by delivery address, etc.). The packing device 226 may include ice or temperature sensitive elements for prescriptions that are to be kept within a temperature range during shipping (for example, this may be necessary in order to retain efficacy). The ultimate package may then be shipped through postal mail, through a mail order delivery service that ships via ground and/or air (e.g., UPS, FEDEX, or DHL, etc.), through a delivery service, through a locker box at a shipping site (e.g., AMAZON locker or a PO Box, etc.), or otherwise.

The unit of use packing device 230 packages a unit of use prescription order in preparation for shipping the order. The unit of use packing device 230 may include manual scanning of containers to be bagged for shipping to verify each container in the order. In an example implementation, the manual scanning may be performed at a manual scanning station. The pharmacy fulfillment device 112 may also include a mail manifest device 232 to print mailing labels used by the packing device 226 and may print shipping manifests and packing lists.

While the pharmacy fulfillment device 112 in FIG. 2 is shown to include single devices 206-232, multiple devices may be used. When multiple devices are present, the multiple devices may be of the same device type or models, or may be a different device type or model. The types of devices 206-232 shown in FIG. 2 are example devices. In other configurations of the system 100, lesser, additional, or different types of devices may be included.

Moreover, multiple devices may share processing and/or memory resources. The devices 206-232 may be located in the same area or in different locations. For example, the devices 206-232 may be located in a building or set of adjoining buildings. The devices 206-232 may be interconnected (such as by conveyors), networked, and/or otherwise in contact with one another or integrated with one another (e.g., at the high-volume fulfillment center, etc.). In addition, the functionality of a device may be split among a number of discrete devices and/or combined with other devices.

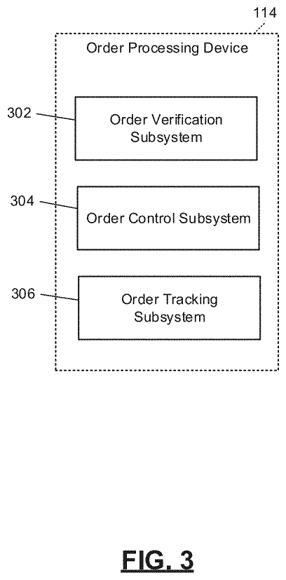

FIG. 3 illustrates the order processing device 114 according to an example implementation. The order processing device 114 may be used by one or more operators to generate prescription orders, make routing decisions, make prescription order consolidation decisions, track literature with the system 100, and/or view order status and other order related information. For example, the prescription order may be comprised of order components.

The order processing device 114 may receive instructions to fulfill an order without operator intervention. An order component may include a prescription drug fulfilled by use of a container through the system 100. The order processing device 114 may include an order verification subsystem 302, an order control subsystem 304, and/or an order tracking subsystem 306. Other subsystems may also be included in the order processing device 114.

The order verification subsystem 302 may communicate with the benefit manager device 102 to verify the eligibility of the member and review the formulary to determine appropriate copayment, coinsurance, and deductible for the prescription drug and/or perform a DUR (drug utilization review). Other communications between the order verification subsystem 302 and the benefit manager device 102 may be performed for a variety of purposes.

The order control subsystem 304 controls various movements of the containers and/or pallets along with various filling functions during their progression through the system 100. In some implementations, the order control subsystem 304 may identify the prescribed drug in one or more than one prescription orders as capable of being fulfilled by the automated dispensing device 214. The order control subsystem 304 may determine which prescriptions are to be launched and may determine that a pallet of automated-fill containers is to be launched.

The order control subsystem 304 may determine that an automated-fill prescription of a specific pharmaceutical is to be launched and may examine a queue of orders awaiting fulfillment for other prescription orders, which will be filled with the same pharmaceutical. The order control subsystem 304 may then launch orders with similar automated-fill pharmaceutical needs together in a pallet to the automated dispensing device 214. As the devices 206-232 may be interconnected by a system of conveyors or other container movement systems, the order control subsystem 304 may control various conveyors: for example, to deliver the pallet from the loading device 208 to the manual fulfillment device 216 from the literature device 228, paperwork as needed to fill the prescription.

The order tracking subsystem 306 may track a prescription order during its progress toward fulfillment. The order tracking subsystem 306 may track, record, and/or update order history, order status, etc. The order tracking subsystem 306 may store data locally (for example, in a memory) or as a portion of the order data 118 stored in the storage device 110.

### Claim Processing

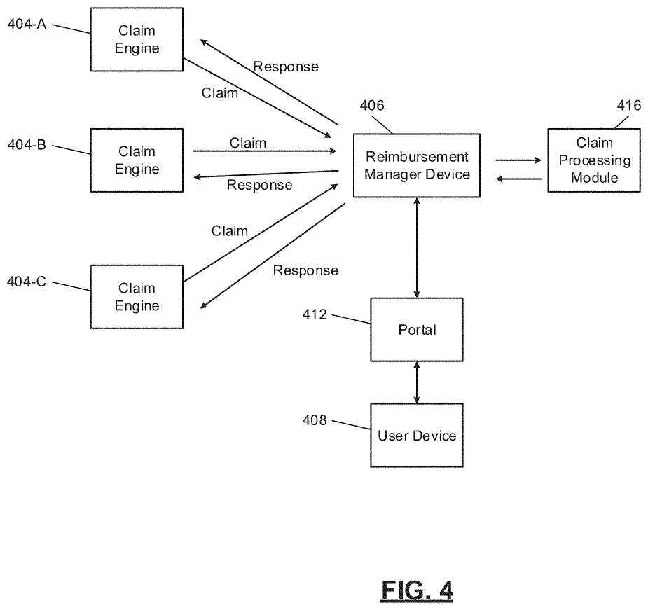

FIG. 4 is a functional block diagram of an example claim processing system. One or more claim engines, such as claim engines 404-A, 404-B, and 404-C (collectively, claim engines 404) transmit claims to a reimbursement manager device 406, such as via the network 104. In various implementations, the reimbursement manager device 406 may be included in the benefit manager device 102. Claim engines may be embodied by one or more processors executing code stored in memory. The claims may be pharmacy claims, healthcare (medical) claims, or both. The claims may include claims for individuals (humans), pets, etc.

The reimbursement manager device 406 determines a type of a claim based on the claim engine from which the claim was received. For example, the reimbursement manager device 406 may determine that a claim is a governmental claim (i.e., a claim for benefits under a governmental benefit plan) when the claim is received from a claim engine in a first predetermined set of one or more claim engines. The reimbursement manager device 406 may determine that a claim is a non-governmental claim (i.e., a claim for benefits that is not under a governmental benefit plan, such as a private benefit plan or another suitable type of benefit plan) when the claim is received from a second predetermined set of one or more claim engines. None of the claim engines in the first predetermined set are also in the second predetermined set.

The reimbursement manager device 406 determines inclusions and exclusions for a claim based on the type of the claim. For example, governmental claims include first inclusions and first exclusions, and non-governmental claims include second inclusions and second exclusions. Inclusions may be rules regarding inclusions of treatments (of claims) for denial, even if those treatments would be approved. Exclusions may be rules regarding approving treatments (of claims), regardless of whether those claims would be denied for other reasons.

Inclusions and exclusions can be updated via user devices, such as user device 408, via a user portal 412. User devices may access the user portal 412 via a network, such as the network 104. Example users that may adjust one or more inclusions and/or exclusions include users of benefit plan providers.

The reimbursement manager device 406 transmits received claims, the determined inclusions and exclusions, and the type of the claim to a claim processing module 416. The reimbursement manager device 406 and the claim processing module 416 may communicate via a network, such as the network 104. An example of a claim processing module includes the ClaimsXten™ claim processing module by McKesson Information Solutions, Inc. While the example of ClaimsXten™ is provided, the present application is also applicable to other types of claim processing modules and code auditing tools (modules).

The claim processing module 416 determines whether to approve or deny claims (or portions of claims) using based on the claim, the received inclusions and exclusions, and predetermined approval/denial rules. The claim processing module 416 responds to the reimbursement manager device 406 with the approval or denial based. The predetermined approval/denial rules may be claim type specific (i.e., one set for governmental claims and one set for non-governmental claims), or the same predetermined approval/denial rules may be applied to governmental and non-governmental claims.

The reimbursement manager device 406 may initiate payment to a provider that submitted a claim when the claim is approved. The reimbursement manager device 406 may deny payment to a provider that submitted a claim when the claim is denied. The reimbursement manager device 406 may also initiate one or more other actions for approved and/or denied claims.

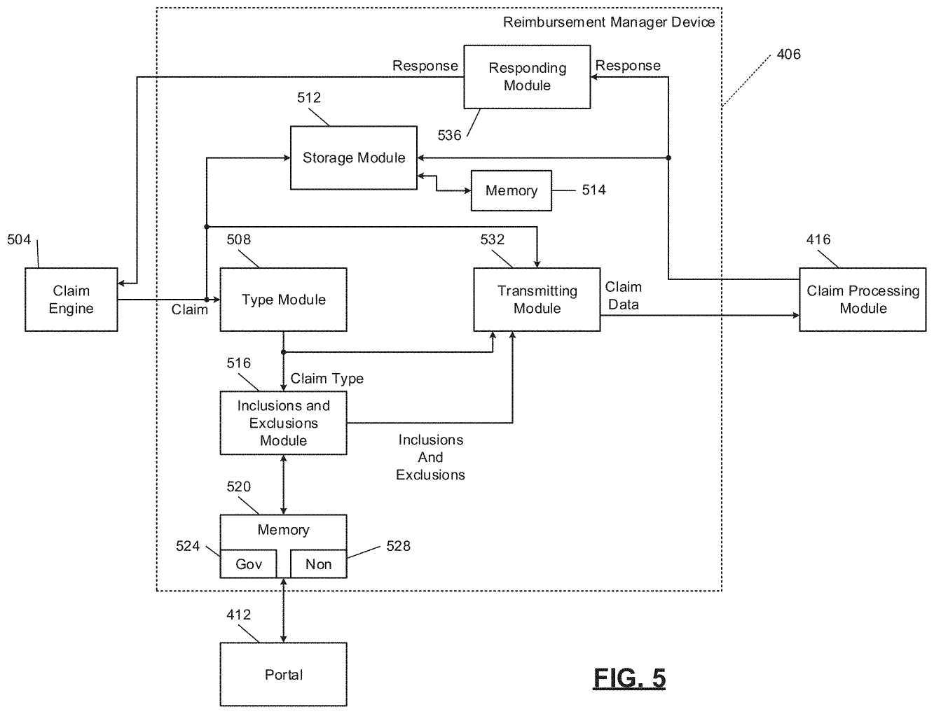

FIG. 5 is a functional block diagram of an example implementation of the reimbursement manager device 406. FIG. 5 illustrates receiving a claim from a claim engine 504. As discussed above, the reimbursement manager device 406 receives claims from multiple claim engines. While the example of one claim will be discussed below, the same may be performed for each received claim. Each claim includes one or more claim codes.

A type module 508 determines a type of the claim (governmental or non-governmental) based on the claim engine from which the claim was received. For example, the type module 508 determines that the claim is a governmental claim when the claim engine (that transmitted the claim) is in the first predetermined set of claim engines (that transmit governmental claims). The type module 508 determines that the claim is a non-governmental when the claim engine (that transmitted the claim) is in the second predetermined set of claim engines. Claim engines in the second predetermined set may transmit only non-governmental claims, and claim engines in the first predetermined set may transmit only governmental claims.

A storage module 512 stores an identifier of the claim in memory 514 when the claim is received. The storage module 512 may store the type of the claim in association with the claim in the memory 514. The storage module 512 also stores other information, as described further below.

An inclusions and exclusions module 516 determines inclusions and exclusions associated with the claim based on the type of the claim. First predetermined inclusions and first predetermined exclusions may be associated with governmental claims and are stored in memory 520. The first predetermined inclusions and the first predetermined exclusions are visually represented by 524 in FIG. 5. Second predetermined inclusions and second predetermined exclusions may be associated with non-governmental claims and are stored in the memory 520. The second predetermined inclusions and the second predetermined exclusions are visually represented by 528 in FIG. 5. Inclusions and exclusions can be changed, such as via the user portal 412, as described above. When the claim is a governmental claim, the inclusions and exclusions module 516 selects the first predetermined inclusions and the first predetermined exclusions as the inclusions and exclusions for the claim. When the claim is a non-governmental claim, the inclusions and exclusions module 516 selects the second predetermined inclusions and the second predetermined exclusions as the inclusions and exclusions for the claim.

A transmitting module 532 transmits claim data for a claim to the claim processing module 416. The claim data includes the claim (including the claim code(s)), the type of the claim, and the inclusions and exclusions for the claim. The claim data may also include other data.

The claim processing module 416 determines whether to approve or deny (e.g., payment of) the claim by comparing the claim code(s) of the claim with the inclusions, exclusions, and predetermined approval/denial rules. For example, the claim processing module 416 may determine to deny the claim when the claim code(s) is/are included in the inclusions and satisfies one or more rules. The claim processing module 416 may determine to approve the claim when the claim code(s) is/are included in the exclusions. This may be true regardless of whether the claim code(s) are included in the claim would be denied according to the predetermined approval/denial rules. If the claim code(s) is/are not included in neither the inclusions nor the exclusions, the claim processing module 416 may determine whether to approve or deny the claim using the predetermined approval/denial rules.

The claim processing module 416 transmits a response to the reimbursement manager device 406 regarding approval or denial of the claim. The response indicates whether the claim processing module 416 determined whether to approve or deny payment of the claim.

The storage module 512 stores the decision (approval or denial) in association with the claim in the memory 514. In this manner, the decisions (approval/denial) of the claim processing module 416 can be checked for accuracy. A responding module 536 transmits the response back to the claim engine 504 that transmitted the claim, such as via the network.

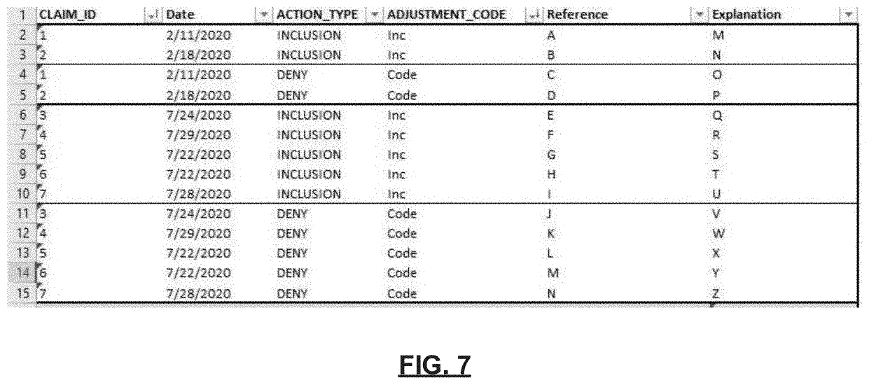

FIG. 7 is an example table including claim identifiers, action dates, action types, adjustment codes, references, and explanations for inclusions. The information in claim 7, along with other information, may be stored in the memory 514. The leftmost column may include the unique identifier of a claim. While numerical identifiers are provided as examples, alpha numeric or other suitable form of unique identifier may be used.

Rows 2 and 3 are example claims received on different dates. Rows 4 and 5 include responses to the claims of rows 2 and 3, respectively. Similarly, rows 6-10 are for example claims. Rows 11-15 are for example responses to the claims of rows 6-10, respectively.

The second column from the left indicates the date of action. This column the receipt date of the claims for rows 2, 3, and 6-10. This column includes the response date for rows 4, 5, and 11-15.

The third row from the left includes action for each row. Inclusion may mean that the claim is subject to an inclusion. Deny indicates that the response will be to deny the claim based on an inclusion.

The second row from the right provides a reference rule set to be used regarding the inclusion. The rightmost column includes an explanation regarding the response after processing based on the inclusion and reference rule set.

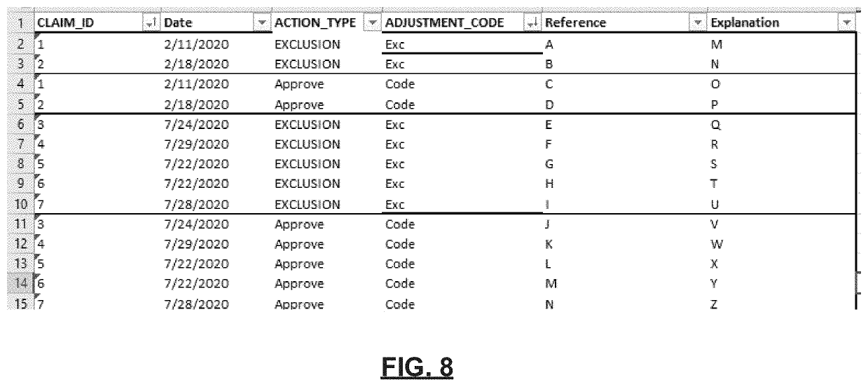

A similar format may be used regarding exclusions. FIG. 8 is an example table including claim identifiers, action dates, action types, adjustment codes, references, and explanations for exclusions.

One or more actions may be taken based on the response. For example, the claim engine 504 may initiate payment of the claim to a submitter (e.g., health care provider) of the claim when the claim is approved. The claim engine 504 may not initiate payment of the claim to the submitter when the claim is denied. The claim engine 504 may transmit one or more indicators of the decision (approval or denial) to one or more users, such as a user computer associated with the submitter. The claim engine 504 may trigger generation and transmission of an EOB to a user associated with the claim. One or more other actions may also be taken.

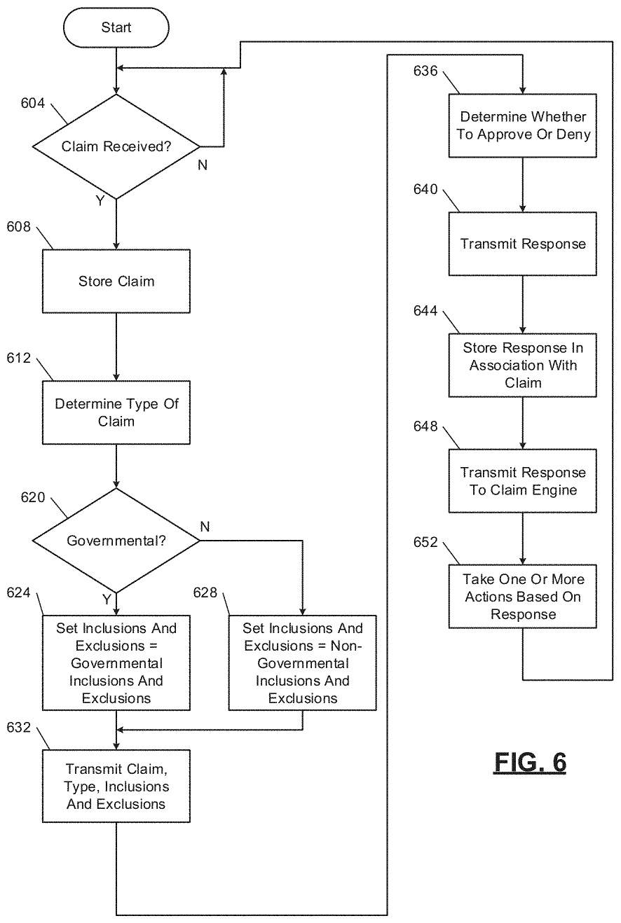

FIG. 6 is a flowchart depicting an example method of processing a claim. The example of FIG. 6 may be performed for each claim received. Control begins with 604 where the reimbursement manager device 406 determines whether a claim has been received from a claim engine. If 604 is true, control continues with 608. If 604 is false, control remains at 604.

At 608, the storage module 512 stores the claim in the memory 514. At 612, the type module 508 determines the type of the claim (governmental or non-governmental) based on the claim engine that transmitted the claim. The type module 508 may determine that the claim is a governmental claim when the claim engine that transmitted the claim is in the first predetermined set of claim engines. The type module 508 may determine that the claim is a non-governmental claim when the claim engine that transmitted the claim is in the second predetermined set of claim engines.

At 620, the inclusions and exclusions module 516 determines whether the claim is a governmental claim. If 620 is true, the inclusions and exclusions module 516 sets inclusions and exclusions for the claims to the first predetermined inclusions and the first predetermined exclusions, respectively, at 624. If 620 is false, the inclusions and exclusions module 516 sets the inclusions and exclusions for the claim to the second predetermined inclusions and the second predetermined exclusions, respectively, at 628.

At 632, the transmitting module 532 transmits the claim, the type of the claim, and the inclusions and exclusions for the claim to the claim processing module 416, such as the ClaimsXten™ claim processing module. At 636, the claim processing module 416 determines whether to approve or deny the claim based on the claim code(s) of the claim and using the inclusions, the exclusions, and the predetermined approval/denial claims, as discussed above.

At 640, the claim processing module 416 transmits the response (including the indication of whether to approve or deny the claim) to the reimbursement manager device 406. At 644, the storage module 512 stores the indication of whether to approve or deny the claim in association with the claim in memory 514. For example only, the memory 514 may include an object oriented database where entries can be associated with one or more other entries.

At 648, the responding module 536 transmits a response including the indication of whether to approve or deny the claim to the claim engine 504 from which the claim was received. At 652, the claim engine may take one or more actions based on the response. For example, the claim engine may generate an EOB for the user and send the EOB to the member, such as to a mailing address associated with the user, to an email address associated with the user, etc. The claim engine may also initiate payment or non-payment of the claim to a provider that submitted the claim. One or more other actions may be taken additionally or alternatively. In the example of the claim being a pharmacy claim, fulfillment (e.g., packing, labeling, etc. discussed above) and shipment of the prescription may be initiated.

### CONCLUSION

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. As used herein, the phrase at least one of A, B, and C should be construed to mean a logical (A OR B OR C), using a non-exclusive logical OR, and should not be construed to mean “at least one of A, at least one of B, and at least one of C.”

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A. The term subset does not necessarily require a proper subset. In other words, a first subset of a first set may be coextensive with (equal to) the first set.

In this application, including the definitions below, the term “module” or the term “controller” or the term “engine” may be replaced with the term “circuit.” The term “module” may refer to, be part of, or include processor hardware (shared, dedicated, or group) that executes code and memory hardware (shared, dedicated, or group) that stores code executed by the processor hardware.

The module may include one or more interface circuits. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the WIFI wireless networking standard) and IEEE Standard 802.3-2015 (also known as the ETHERNET wired networking standard). Examples of a WPAN are the BLUETOOTH wireless networking standard from the Bluetooth Special Interest Group and IEEE Standard 802.15.4.

The module may communicate with other modules using the interface circuit(s). Although the module may be depicted in the present disclosure as logically communicating directly with other modules, in various implementations the module may actually communicate via a communications system. The communications system includes physical and/or virtual networking equipment such as hubs, switches, routers, and gateways. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the module may be distributed among multiple modules that are connected via the communications system. For example, multiple modules may implement the same functionality distributed by a load balancing system. In a further example, the functionality of the module may be split between a server (also known as remote, or cloud) module and a client (or, user) module.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of a non-transitory computer-readable medium are nonvolatile memory devices (such as a flash memory device, an erasable programmable read-only memory device, or a mask read-only memory device), volatile memory devices (such as a static random access memory device or a dynamic random access memory device), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Swift, Haskell, Go, SQL, R, Lisp, Java®, Fortran, Perl, Pascal, Curl, OCaml, JavaScript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

What is claimed is:

1. A processing system, comprising:
a type module configured to receive a claim from a claim engine and to determine a type of the claim based on the claim engine from which the claim was received;
an inclusions and exclusions module configured to, based on the type of the claim, select inclusions and exclusions for the claim;
a transmitting module configured to transmit, to a claim processing module, (a) the claim, (b) the selected inclusions and exclusions, and (c) the type of the claim; and
a responding module configured to:
receive, from the claim processing module, a response including an indicator of whether to (a) approve the claim or (b) deny the claim, the response being determined by the claim processing module based on the claim, the selected inclusions and exclusions, the type of the claim, and rules regarding approving and denying claims; and
to respond to the claim engine with the indicator of whether to (a) approve the claim or (b) deny the claim.

2. The processing system of claim 1 wherein the type is selected from a group consisting of: governmental and non-governmental.

3. The processing system of claim 1 wherein the type module is configured to:
set the type to governmental when the claim engine is included in a first predetermined set of claim engines; and
set the type to non-governmental when the claim engine is included in a second predetermined set of claim engines,
wherein none of the claim engines in the second predetermined set of claim engines is included in the first predetermined set of claim engines.

4. The processing system of claim 1 wherein the inclusions and exclusions module is configured to:
select first inclusions and first exclusions when the type of the claim is governmental; and
select second inclusions and second exclusions when the type of the claim is non-governmental.

5. The processing system of claim 4 wherein:
at least one of the first inclusions is not included in the second inclusions; and
at least one of the second inclusions is not included in the first inclusions.

6. The processing system of claim 4 wherein:
at least one of the first exclusions is not included in the second exclusions; and
at least one of the second exclusions is not included in the first exclusions.

7. The processing system of claim 1 wherein the transmitting module is configured to transmit (a) the claim, (b) the selected inclusions and exclusions, and (c) the type of the claim to the claim processing module via a network.

8. The processing system of claim 1 wherein the type module is configured to receive the claim from the claim engine via a network.

9. The processing system of claim 1 further comprising a storage module configured to store the claim in memory, to store the type of the claim in the memory in association with the claim, and to store, in association with the claim, the indicator of whether to (a) approve the claim or (b) deny the claim.

10. The processing system of claim 1 wherein the claim includes a claim code for a healthcare procedure performed by a health care provider.

11. The processing system of claim 1 wherein the claim processing module includes a CLAIMSXTEN claim processing module.

12. The processing system of claim 1 wherein the selected inclusions include rules for denying claims and the selected exclusions include rules for approving claims.

13. A processing method, comprising:
receiving a claim from a claim engine;
determining a type of the claim based on the claim engine from which the claim was received;
based on the type of the claim, selecting inclusions and exclusions for the claim;
transmitting, to a claim processing module, (a) the claim, (b) the selected inclusions and exclusions, and (c) the type of the claim;
receiving, from the claim processing module, a response including an indicator of whether to (a) approve the claim or (b) deny the claim, the response being determined by the claim processing module based on the claim, the selected inclusions and exclusions, the type of the claim, and rules regarding approving and denying claims; and
responding to the claim engine with the indicator of whether to (a) approve the claim or (b) deny the claim.

14. The processing method of claim 13 wherein the type is selected from a group consisting of: governmental and non-governmental.

15. The processing method of claim 13 further comprising:
setting the type to governmental when the claim engine is included in a first predetermined set of claim engines; and
setting the type to non-governmental when the claim engine is included in a second predetermined set of claim engines,
wherein none of the claim engines in the second predetermined set of claim engines is included in the first predetermined set of claim engines.

16. The processing method of claim 13 wherein the selecting inclusions and exclusions includes:
selecting first inclusions and first exclusions when the type of the claim is governmental; and
selecting second inclusions and second exclusions when the type of the claim is non-governmental.

17. The processing method of claim 16 wherein:
at least one of the first inclusions is not included in the second inclusions; and
at least one of the second inclusions is not included in the first inclusions.

18. The processing method of claim 16 wherein:
at least one of the first exclusions is not included in the second exclusions; and
at least one of the second exclusions is not included in the first exclusions.

19. The processing method of claim 13 further comprising:
storing the claim in memory;
storing the type of the claim in the memory in association with the claim; and
storing, in association with the claim, the indicator of whether to (a) approve the claim or (b) deny the claim.

20. The processing method of claim 13 wherein the selected inclusions include rules for denying claims and the selected exclusions include rules for approving claims.
